SEQUENCE LISTING (14 sequences)

SEQ ID NO: 1 - protein, 12 aa, Artificial Sequence
  Synthetic
MGESDDSILRLA

SEQ ID NO: 2 - protein, 15 aa, Artificial Sequence
  Synthetic
FLVTEVENGGSLGSK

SEQ ID NO: 3 - protein, 19 aa, Artificial Sequence
  Synthetic
PEPAKSAPAPKKGSKKAVT

SEQ ID NO: 4 - protein, 14 aa, Artificial Sequence
  Synthetic
VSPAAGSSPGKPPR

SEQ ID NO: 5 - protein, 18 aa, Artificial Sequence
  Synthetic
AAGKGPLATGGIKKSGKK

SEQ ID NO: 6 - protein, 12 aa, Artificial Sequence
  Synthetic
LRPAVSSSDQQS

SEQ ID NO: 7 - protein, 11 aa, Artificial Sequence
  Synthetic
ISHELDSASSE

SEQ ID NO: 8 - protein, 20 aa, Artificial Sequence
  Synthetic
KANDESNEHSDVIDSQELSK

SEQ ID NO: 9 - protein, 19 aa, Artificial Sequence
  Synthetic
KLEKEEEEGISQESSEEEQ

SEQ ID NO: 10 - protein, 13 aa, Artificial Sequence
  Synthetic
RSAPGGGSKVPQK

SEQ ID NO: 11 - protein, 21 aa, Artificial Sequence
  Synthetic
RSGPTDDGEEEMEEDTVTNGS

SEQ ID NO: 12 - protein, 18 aa, Artificial Sequence
  Synthetic
TVAGGAWTYNTTSAVTVK

SEQ ID NO: 13 - protein, 19 aa, Artificial Sequence
  Synthetic
VIYTRNTKGGDAPAAGEDA

SEQ ID NO: 14 - protein, 14 aa, Artificial Sequence
  Synthetic
YTTAEEAAGIGILT

(19) United States
(12) Patent Application Publication
(10) Pub. No.: US 2023/0381291 A1
(43) Pub. Date: Nov. 30, 2023

(54) TUMOR-ASSOCIATED PEPTIDES AND USES THEREOF

(71) Applicants: HUMANITAS MIRASOLE S.P.A., Rozzano (MI) (IT); HUMANITAS UNIVERSITY, Pieve Emanuele (MI) (IT); ISTITUTO EUROPEO DI ONCOLOGIA S.R.L., Milano (MI) (IT)

(72) Inventors: Maria RESCIGNO, Milano (MI) (IT); Alessia MELACARNE, Milano (MI) (IT); Giuseppe PENNA, Cusano Milanino (MI) (IT)

(73) Assignees: HUMANITAS MIRASOLE S.P.A., Rozzano (MI) (IT); HUMANITAS UNIVERSITY, Pieve Emanuele (MI) (IT); ISTITUTO EUROPEO DI ONCOLOGIA S.R.L., Milano (MI) (IT)

(21) Appl. No.: 18/004,831

(22) PCT Filed: Jul. 8, 2021
(86) PCT No.: PCT/EP2021/068942

§ 371 Date: Jan. 9, 2023

(30) Foreign Application Priority Data
Jul. 10, 2020 (IT) 102020000016807

(51) Int. Cl. A61K 39/00; A61P 37/04

(52) U.S. Cl. CPC A61K 39/0011; A61P 37/04; A61K 2039/5154; A61K 2039/876

(57) ABSTRACT

The present invention provides antigen presenting cells (APC) carrying human tumor-associated peptides on the cell surface as well as immunogenic compositions including the tumor-associated peptides and/or the antigen presenting cells according to the invention. The immunogenic composition of the invention is useful as a vaccine in the prevention and/or treatment of a tumor disease, particularly melanoma and melanoma residual disease.

The present invention lies within the field of immunotherapy, particularly within the field of immunotherapy for neoplastic pathologies.

Immunotherapy and, in particular, immune checkpoint inhibitors (ICB), have drastically improved patients' overall survival in highly immunogenic solid tumors such as metastatic melanoma and lung cancer (Vanpouille-box et al. 2017). However, a large proportion of patients do not respond to therapy (Wolchok et al. 2013). Failure of ICB efficacy is mostly due to the absence of a pre-existing antitumor response that can be boosted or revitalized by the immune check point inhibitors (Gros et al. 2014) and/or to the inability of tumor cells to present tumor-derived peptides (Gao et al. 2016; Zaretsky et al. 2016). The current view is that combinatorial approaches aimed at both activating the immune response and protecting it from tumor-evasion strategies may represent a valid alternative for treating non-responding patients (Sharma and Allison 2015). Consistently, two clinical trials have shown that therapeutic vaccination based on patient-specific neoantigens can elicit an anti-tumor response in advanced metastatic melanoma and that this can be boosted using a combination with the ICB anti-PD1 (Ott et al. 2017; Sahin et al. 2017). However, the same seems not to be true when analyzing the combination of ICB with only one known melanoma antigenic peptide (such as glycoprotein gp100, Hodi et al. 2010) suggesting that the nature of the antigen is fundamental for achieving an effective antitumor immunity. Indeed, tumor cells with a high mutational load generate a series of novel antigens that carry somatic mutations which, following the antigen processing and presentation pathway, are then presented by the HLA class I (HLA-I) and elicit a CD8+ cytotoxic T cell (CTL) response. Typically, such novel antigens deriving from mutated proteins, also called neoantigens, are unique for a particular patient (Palmieri et al., 2017; Snyder et al., 2014.).

Despite being very attractive, a personalized vaccine strategy based on the identification of patient-specific neoantigens, is time consuming, expensive, and difficult to translate to all cancer centers and individual patients.

The researches described in Saccheri F. et al, “Bacteria-induced gap junctions in tumors favor antigen cross-presentation and antitumor immunity”, (2010), Sci Transl Med. 11; 2(44):44ra57, revealed that the infection of murine and human tumor cells with the attenuated vaccine strain of Salmonella typhi leads to the overexpression of Cx43, the most abundant and ubiquitous component of plasma membrane hemichannels forming gap junctions (GJ). Bacteria-treated tumor cells establish gap junctions with dendritic cells (DCs) by the docking of two plasma membrane hemichannels, allowing the transfer of pre-processed antigens from tumor cells to DCs, and this leads to the establishment of a strong DC-mediated antitumor response. This strategy is however quite laborious to translate to clinical application since it requires the simultaneous generation of a tumor cell line, the differentiation of DCs from peripheral blood monocytes (PBMCs), and the pairing of tumor cells with DCs from patient biomaterial.

WO 2017/081286 A1 discloses that the cell culture supernatant of Salmonella-infected human, canine and murine tumor cells exhibits immunogenic potential. It also discloses some murine peptides isolated from the supernatant of Salmonella-infected murine tumor cells.

In the light of the foregoing, there is a need for a novel and effective immunotherapy approach in cancer treatment. More particularly, there is a need for a cancer-targeted immunotherapy approach suitable of clinical application across a broad patient population and exhibiting at the same time reduced adverse side effects, more specifically reduced autoimmunity reactions.

These and other needs are met by the isolated antigen presenting cell (APC), the immunogenic composition and the therapeutic uses thereof as defined in the appended claims, which form an integral part of the description.

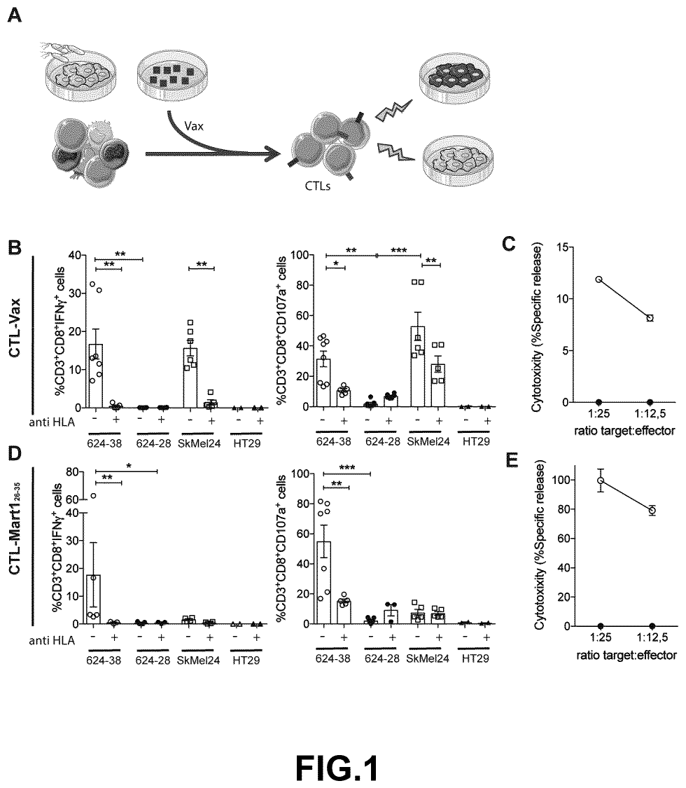

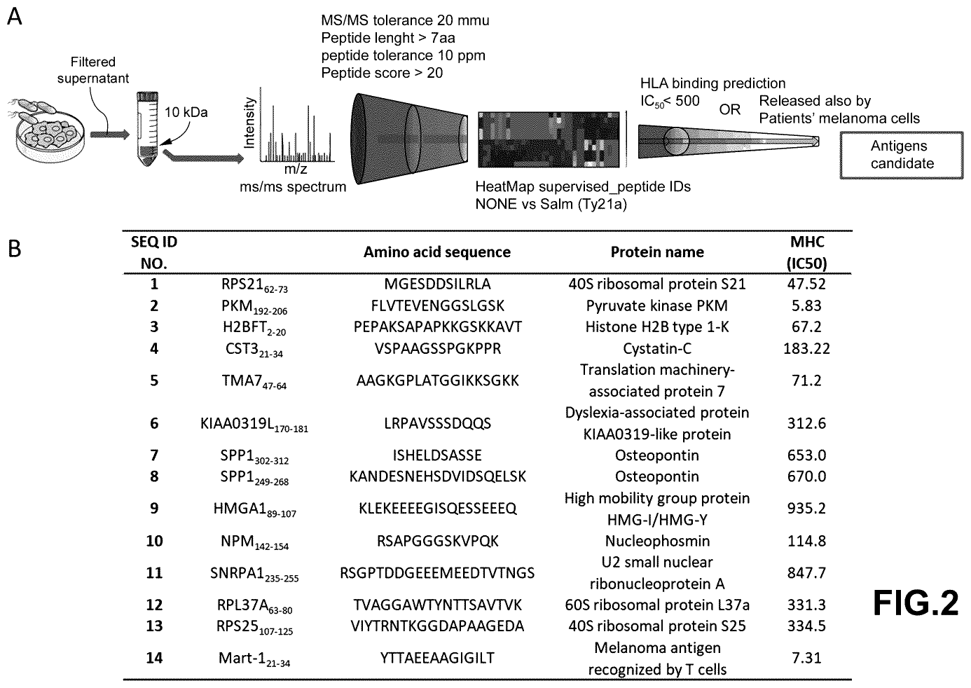

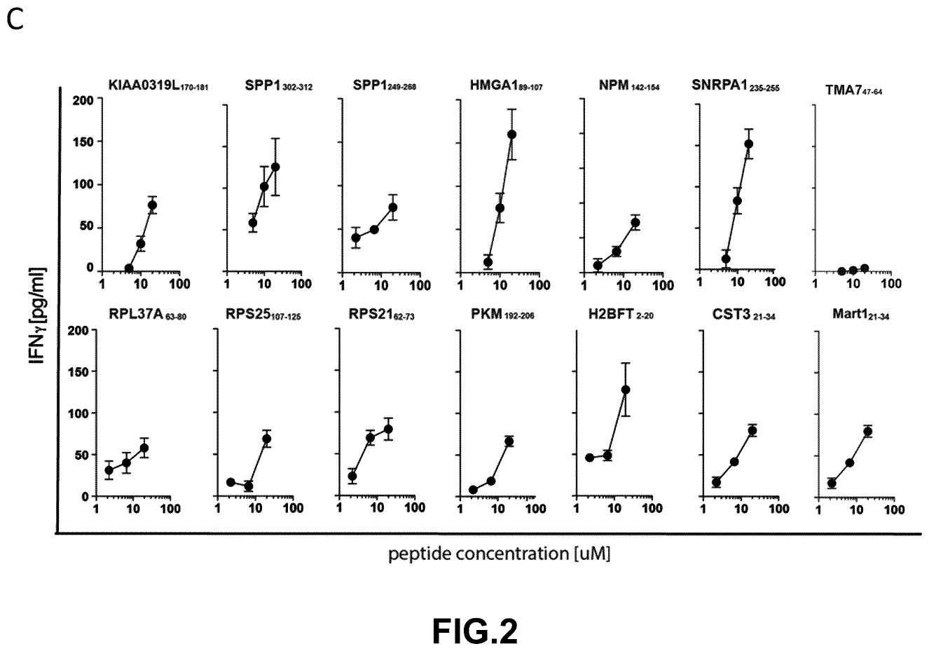

The present inventors succeed in obtaining for the first time novel peptides released from tumor cells, which are surprisingly capable of activating, either alone or in combination, tumor specific-cytotoxic T-lymphocytes (CTLs) towards the killing of tumor cells (FIG. 2). As it will be illustrated in detail in the Experimental section below, the peptides of the invention were isolated from the proteins secreted by bacteria-infected tumor cells, in particular Salmonella-infected human melanoma cells, and are shared among tumor cells from different patients affected by the same pathology as well as among different cells from the same tumor lesion (FIG. 1).

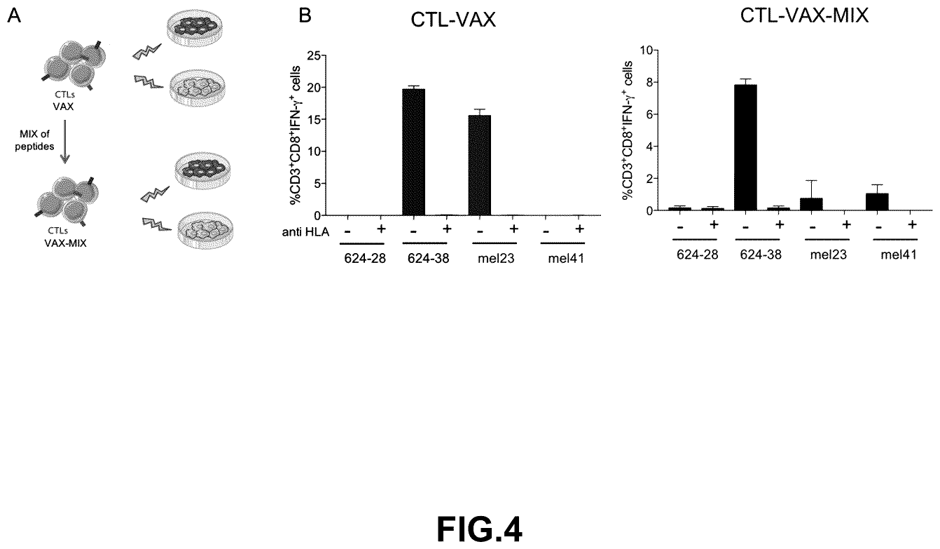

Moreover, the studies conducted by the present inventors surprisingly revealed that CTLs stimulated with the peptides of the invention are not activated by primary melanocytes, thereby demonstrating that these peptides are neither produced nor presented by normal healthy cells (FIG. 4).

The present inventors have indeed found that the peptides of the invention are processed by the tumor proteasome machinery via the unfolded protein response (UPR), which is as an adaptive mechanism intrinsic and unique of cancer cells. Typically, the UPR pathway is activated in cancer cells in response to the stress stimuli these highly proliferating cells have to sustain at the level of the endoplasmic reticulum. Without wishing to be bound by any theory, the present inventors believe that bacterial infection may exacerbate UPR activation in ER-stressed tumor cells by acting as an additional stress-stimulus.

Therefore, the peptides obtained by the present inventors represent a unique antigen signature capable of exerting a highly specific immunogenic effect against a tumor, particularly melanoma, in a wide range of patients, without affecting patients' healthy cells. Such unique properties make the peptides of the invention particularly suitable for broad applications in immunotherapy approaches aiming at efficiently targeting different patients with very little or no risk of nonspecific autoimmunity reactions, thereby overcoming the limitations and major hurdles of prior art personalized immunotherapy approaches which need to be tailored to each patient.

As is known in the art, antigen presentation describes a vital immune process which is essential for triggering T cell immune response. Because T cells recognize only fragmented antigens displayed on cell surfaces, antigen processing is typically carried out by antigen-presenting cells (APC) that break a protein antigen into peptides, and present it in conjunction with class II MHC molecules on the cell surface where it may be recognized by a T cell receptor. However, APCs constitutively express a different type of proteasome than tumor cells and the processed peptides present on HLA molecules on APCs may not necessarily correspond to the peptides presented by the tumor cells, thus failing in prompting an effective antitumor response (Vigneron & Van den Eynde; “Proteasome subtypes and the processing of tumor antigens: increasing antigenic diversity” (2012) Curr Opin Immunol. 2012 February; 24(1):84-91).

Advantageously, the tumor-associated peptides of the invention are pre-processed by the tumor proteasome and further trimming of these peptides is not required when they are taken up by APCs to be presented for T lymphocytes recognition. This requisite allows the peptide-loaded APCs to prime efficient T cell responses against tumor cells expressing the same antigens.

A first aspect of the present invention is therefore an isolated antigen-presenting cell (APC), which carries on the cell surface one or more tumor-associated peptides comprising an amino acid sequence selected from the group consisting of SEQ ID NOs. 1-4 and 6-13, fragments of said amino acid sequences SEQ ID Nos. 1-4 and 6-13 of at least 3 amino acids in length, and any combination thereof.

According to the invention, the one or more isolated tumor-associated peptides comprise or consist of or consist essentially of an amino acid sequence selected from SEQ ID NOs. 1-4 and 6-13 and fragments of said amino acid sequences SEQ ID NOs. 1-4 and 6-13 of at least 3 amino acids in length.

In a preferred embodiment, the one or more isolated peptides of the invention are melanoma-associated peptides, preferably peptides associated with human melanoma.

As used herein, the term “fragment” refers to a continuous sequence of amino acid residues, which sequence forms a subset of a larger amino acid sequence.

In one embodiment, said fragments of amino acid sequences SEQ ID NOs. 1-4 and 6-13 are at least 4, at least 5, at least 6, at least 7, at least 8, at least 9, at least 10, at least 11, at least 12, at least 13, at least 14, at least 15, at least 16, at least 17, at least 18, at least 19, at least 20 or at least 21 amino acids in length.

In another embodiment, said fragments of amino acid sequences SEQ ID NOs. 1-4 and 6-13 are 3, 4, 5, 6, 7, 8, 9, 10, 11, 12, 13, 14, 15, 16, 17, 18, 19, 20 or 21 amino acids in length.

In a preferred embodiment, said fragments of amino acid sequences SEQ ID NOs. 1-4 and 6-13 are 8 amino acids in length.

In a more preferred embodiment, said fragments are capable of triggering an immune response, more preferably a cell-mediated immune response, even more preferably a T cell-mediated immune response against tumor cells, including for example melanoma cells.

In one embodiment, the antigen presenting cell (APC) carries on the cell surface one or more tumor-associated peptides comprising or consisting of an amino acid sequence of either SEQ ID NOs. 1, 2, 3 or 4, and any combination thereof.

According to this embodiment, the antigen presenting cell (APC) preferably carries on the cell surface a tumor-associated peptide comprising or consisting of SEQ ID NO. 1, a tumor-associated peptide comprising or consisting of SEQ ID NO. 2, a tumor-associated peptide comprising or consisting of SEQ ID NO. 3 and a tumor-associated peptide comprising or consisting of SEQ ID NO. 4.

In another embodiment, the antigen presenting cell (APC) carries on the cell surface one or more tumor-associated peptides comprising or consisting of an amino acid sequence of either SEQ ID NOs. 6, 7, 8, or 9, and any combination thereof.

According to this embodiment, the antigen presenting cell (APC) preferably carries on the cell surface a tumor-associated peptide comprising or consisting of SEQ ID NO. 6, a tumor-associated peptide comprising or consisting of SEQ ID NO. 7, a tumor-associated peptide comprising or consisting of SEQ ID NO. 8 and a tumor-associated peptide comprising or consisting of SEQ ID NO. 9.

In a still another embodiment, the antigen presenting cell (APC) carries on the cell surface a tumor-associated peptide comprising or consisting of SEQ ID NO. 1, a tumor-associated peptide comprising or consisting of SEQ ID NO. 2, a tumor-associated peptide comprising or consisting of SEQ ID NO. 3, a tumor-associated peptide comprising or consisting of SEQ ID NO. 4, a tumor-associated peptide comprising or consisting of SEQ ID NO. 6, a tumor-associated peptide comprising or consisting of SEQ ID NO. 7, a tumor-associated peptide comprising or consisting of SEQ ID NO. 8, and a tumor-associated peptide comprising or consisting of SEQ ID NO. 9.

In a further embodiment, the antigen presenting cell (APC) carries on the cell surface a tumor-associated peptide comprising or consisting of SEQ ID NO. 1, a tumor-associated peptide comprising or consisting of SEQ ID NO. 2, a tumor-associated peptide comprising or consisting of SEQ ID NO. 3, a tumor-associated peptide comprising or consisting of SEQ ID NO. 4, a tumor-associated peptide comprising or consisting of SEQ ID NO. 7, a tumor-associated peptide comprising or consisting of SEQ ID NO. 8, a tumor-associated peptide comprising or consisting of SEQ ID NO. 11, a tumor-associated peptide comprising or consisting of SEQ ID NO. 12 and a tumor-associated peptide comprising or consisting of SEQ ID NO. 13.

In a still further embodiment, the antigen presenting cell (APC) carries on the cell surface a tumor-associated peptide comprising or consisting of SEQ ID NO. 1, a tumor-associated peptide comprising or consisting of SEQ ID NO. 2, a tumor-associated peptide comprising or consisting of SEQ ID NO. 3, a tumor-associated peptide comprising or consisting of SEQ ID NO. 4, a tumor-associated peptide comprising or consisting of SEQ ID NO. 6, a tumor-associated peptide comprising or consisting of SEQ ID NO. 7, a tumor-associated peptide comprising or consisting of SEQ ID NO. 8, a tumor-associated peptide comprising or consisting of SEQ ID NO. 9, a tumor-associated peptide comprising or consisting of SEQ ID NO. 10, a tumor-associated peptide comprising or consisting of SEQ ID NO. 11, a tumor-associated peptide comprising or consisting of SEQ ID NO. 12 and a tumor-associated peptide comprising or consisting of SEQ ID NO. 13.

Preferably, the antigen presenting cell (APC) according to the invention is a dendritic cell (DC).

Dendritic cells can be obtained from any source and may be autologous or allogeneic. As used herein, a cell that is “autologous” to a subject means the cell was isolated from the subject or derived from a cell that was isolated from the subject.

In order to generate an antigen-presenting cell, preferably a dendritic cell, which carries on the cell surface one or more tumor-associated peptides as above defined, autologous or allogenic antigen-presenting cells may be transfected with an expression vector which produces said tumor-associated peptide(s).

According to an alternative antigen loading strategy, an antigen-presenting cell, preferably a dendritic cell, may be pulsed with one or more tumor-associated peptides as above defined.

The skilled person will be aware of techniques and methods for generating an antigen-presenting cell, preferably a dendritic cell, which carries on the cell surface one or more tumor-associated peptides of the invention, and any such suitable method may be used.

A second aspect of the present invention is an immunogenic composition comprising
(i) one or more tumor-associated peptides comprising an amino acid sequence selected from the group consisting of SEQ ID NOs. 1-4 and 6-13, fragments of said amino acid sequences SEQ ID Nos. 1-4 and 6-13 of at least 3 amino acids in length, and any combination thereof;
(ii) one or more isolated nucleic acid sequences encoding the tumor-associated peptide(s) of (i);
(iii) one or more expression vectors comprising the nucleic acid sequence(s) of (ii); and/or
(iv) one or more antigen-presenting cells (APC) as above defined, and a pharmaceutically acceptable vehicle.

As used herein, the term “immunogenic” refers to the ability to stimulate the immune system and elicit/induce an immune response in a host or subject. The expressions “elicit an immune response” or “induce an immune response” both refer to the stimulation of immune cells in vivo in response to a stimulus, such as an antigen. The immune response consists of both cellular immune responses, e.g., T cell, such as cytotoxic T lymphocytes, and macrophage stimulation, and humoral immune response, e.g., B cell and complement stimulation and antibody production. Immune response may be measured using techniques well-known in the art, including, but not limited to, antibody immunoassays, proliferation assays, and others.

According to the invention, any combination of the tumor-associated peptides as above defined may be comprised in the immunogenic composition of the invention.

In one embodiment, the immunogenic composition of the invention comprises one or more tumor-associated peptides comprising or consisting of an amino acid sequence of either SEQ ID NOs. 1, 2, 3 or 4, and any combination thereof.

According to this embodiment, the immunogenic composition of the invention preferably comprises a tumor-associated peptide comprising or consisting of SEQ ID NO. 1, a tumor-associated peptide comprising or consisting of SEQ ID NO. 2, a tumor-associated peptide comprising or consisting of SEQ ID NO. 3 and a tumor-associated peptide comprising or consisting of SEQ ID NO. 4.

In another embodiment, the immunogenic composition of the invention comprises one or more tumor-associated peptides comprising or consisting of an amino acid sequence of either SEQ ID NOs. 6, 7, 8, or 9, and any combination thereof.

According to this embodiment, the immunogenic composition of the invention preferably comprises a tumor-associated peptide comprising or consisting of SEQ ID NO. 6, a tumor-associated peptide comprising or consisting of SEQ ID NO. 7, a tumor-associated peptide comprising or consisting of SEQ ID NO. 8 and a tumor-associated peptide comprising or consisting of SEQ ID NO. 9.

In a still another embodiment, the immunogenic composition of the invention comprises a tumor-associated peptide comprising or consisting of SEQ ID NO. 1, a tumor-associated peptide comprising or consisting of SEQ ID NO. 2, a tumor-associated peptide comprising or consisting of SEQ ID NO. 3, a tumor-associated peptide comprising or consisting of SEQ ID NO. 4, a tumor-associated peptide comprising or consisting of SEQ ID NO. 6, a tumor-associated peptide comprising or consisting of SEQ ID NO. 7, a tumor-associated peptide comprising or consisting of SEQ ID NO. 8, and a tumor-associated peptide comprising or consisting of SEQ ID NO. 9.

In a further embodiment, the immunogenic composition of the invention comprises a tumor-associated peptide comprising or consisting of SEQ ID NO. 1, a tumor-associated peptide comprising or consisting of SEQ ID NO. 2, a tumor-associated peptide comprising or consisting of SEQ ID NO. 3, a tumor-associated peptide comprising or consisting of SEQ ID NO. 4, a tumor-associated peptide comprising or consisting of SEQ ID NO. 7, a tumor-associated peptide comprising or consisting of SEQ ID NO. 8, a tumor-associated peptide comprising or consisting of SEQ ID NO. 11, a tumor-associated peptide comprising or consisting of SEQ ID NO. 12 and a tumor-associated peptide comprising or consisting of SEQ ID NO. 13.

In a still further embodiment, the immunogenic composition of the invention comprises a tumor-associated peptide comprising or consisting of SEQ ID NO. 1, a tumor-associated peptide comprising or consisting of SEQ ID NO. 2, a tumor-associated peptide comprising or consisting of SEQ ID NO. 3, a tumor-associated peptide comprising or consisting of SEQ ID NO. 4, a tumor-associated peptide comprising or consisting of SEQ ID NO. 6, a tumor-associated peptide comprising or consisting of SEQ ID NO. 7, a tumor-associated peptide comprising or consisting of SEQ ID NO. 8, a tumor-associated peptide comprising or consisting of SEQ ID NO. 9, a tumor-associated peptide comprising or consisting of SEQ ID NO. 10, a tumor-associated peptide comprising or consisting of SEQ ID NO. 11, a tumor-associated peptide comprising or consisting of SEQ ID NO. 12 and a tumor-associated peptide comprising or consisting of SEQ ID NO. 13.

In the immunogenic composition of the invention, the one or more isolated nucleic acid sequences encoding the tumor-associated peptide(s) as above-defined can be single or double stranded. Nucleic acids of the present invention may be produced by methods well known in the art, such as e.g. recombinant DNA methods.

Alternatively, the nucleic acid encoding a tumor-associated peptide according to the invention may be a synthetic nucleic acid in which the codons have been optimized for increased expression in the host cell in which it is produced. The degeneracy of the genetic code permits variations of the nucleotide sequence, while still producing a peptide having the identical amino acid sequence as the peptide encoded by the native DNA sequence. Nucleic acid sequences possessing a substantially different codon usage while encoding a peptide according to the invention having the same amino acid sequence are encompassed by the present invention.

The one or more expression vectors in the immunogenic composition, comprising the nucleic acid sequence(s) as above defined, may optionally further comprise a promoter sequence and a polyadenylation signal sequence.

Recombinant expression vectors for use in the manufacture of peptides or proteins are known and described in the state of the art, therefore the selection and use thereof are well within the skills of those of average skill in the art. Such vectors can be prokaryotic or eukaryotic vectors. By way of non-limiting example, vectors for expression in prokaryotic cells such as the pQE vector and the pBAD vector are mentioned.

Alternatively, the one or more expression vectors in the immunogenic composition according to the invention may be suitable for expression in eukaryotic cells, for example a transduction system based on the use of a lentiviral vector, an adenovirus vector, a retroviral vector, a baculovirus.

In one embodiment, the eukaryotic cells transfected with the one or more expression vectors of the immunogenic composition are resident cells in target tissues or anatomical regions of an animal, preferably a human being, such as for example dendritic cells, macrophages, and B cells, thereby resulting in the in vivo expression of the one or more vectors.

In another embodiment, the cells transformed or transfected with the one or more expression vectors of the immunogenic composition are in vitro cell or tissue cultures. The cell system used for the expression of the expression vector of the invention can be selected from prokaryotic systems, for example E. coli bacterial cells, or from eukaryotic systems, for example insect cells.

Accordingly, a method of producing a peptide according to the invention comprises culturing a transformed host cell under suitable conditions and for a time sufficient for the expression of the peptide.

Typically, suitable growth conditions depend on the cellular system used and may relate, for example, to the composition of the culture medium, the pH, the relative humidity, the gaseous component, as well as the temperature. The selection of the most suitable cell culturing conditions to be employed in a method of producing a peptide according to the invention falls within the ability of the person skilled in the art.

Optionally, the above method further comprises the step of recovering the produced peptide from the cell culture. The recovery step can be carried out by using protein purification techniques known in the art, for example by protein denaturation, solubilization and/or renaturation, or by one or more chromatographic and/or desalting steps or, alternatively, by ultrafiltration, dialysis and/or freeze-drying.

Alternatively, the tumor-associated peptide according to the invention may be produced by means of synthetic techniques, for example by solid phase peptide synthesis (SPPS) or solution phase synthesis (SPS). The skilled person will be aware of techniques and methods for peptide synthesis; and any such suitable method may be used.

According to the invention, it is envisaged that the one or more tumor-associated peptides, the one or more nucleic acid sequences, the one or more expression vectors and/or the one or more APC cells all as above defined may be present in the immunogenic composition of the invention in any possible combination.

According to the above-described embodiments, the invention provides an immunogenic composition comprising multiple tumor-specific antigens shared by different tumor cells, which has the advantage that tumor heterogeneity, particularly melanoma heterogeneity, can be targeted, which may occur both as interpatient variability and as intra-patient variability arising within the same lesion, or in different lesions of the same patient.

In addition to the above-mentioned tumor-associated peptides, nucleic acid sequences, expression vectors and/or antigen-presenting cells (APC), the immunogenic composition according to the invention comprises a pharmaceutically acceptable vehicle.

The term “pharmaceutically acceptable” refers to compounds which may be administered to mammals without undue toxicity at concentrations consistent with effective activity of the active ingredient. Preferably, the pharmaceutically acceptable vehicle suitable for use in the immunogenic composition of the invention is an aqueous vehicle, such as, for example, an aqueous solution, a saline solution, a Phosphate-buffered saline (PBS) solution, a Hank's Balanced Salt Solution (HBSS) and a ringer's lactate.

The immunogenic composition according to the invention can be prepared in the form of a liquid, frozen suspension, or in a lyophilized form. For this purpose, the immunogenic composition prepared according to the present disclosure further contains a pharmaceutically acceptable carrier and/or diluent. Carriers include, but are not limited to, stabilizers, preservatives, and buffers. Suitable stabilizers are, for example SPGA, Tween compositions, carbohydrates (such as sorbitol, mannitol, starch, sucrose, dextran, glutamate, or glucose), proteins (such as dried milk serum, albumin, or casein), or degradation products thereof. Examples of suitable buffers include alkali metal phosphates. Suitable preservatives include thimerosal, merthiolate, and gentamicin. Diluents include water, aqueous buffer (such as buffered saline), alcohols, and polyols (such as glycerol).

The selection of a pharmaceutically acceptable vehicle, carrier and/or diluent suitable for the immunogenic composition of the invention can be determined by a person of ordinary skill in the art by using his/her normal knowledge.

Thanks to the above illustrated properties of the tumor-associated peptides of the invention, the immunogenic composition according to the present invention is particularly suitable for use as a vaccine, particularly in the prevention and/or therapeutic treatment of a tumor, particularly a solid tumor, more particularly melanoma.

The term “vaccine” as used herein refers to a immunogenic composition as described herein, which is useful to establish in a subject a protective immune response against a tumor, particularly melanoma.

According to the invention, it is contemplated that an adjuvant can be added to the immunogenic composition for use as a vaccine, to efficiently induce humoral immune responses and cell-mediated immunity.

Illustrative, non-limiting examples of adjuvants suitable for use in the immunogenic composition of the invention are complete Freund's adjuvant, incomplete Freund's adjuvant, mineral gels such as aluminum hydroxide, surface active substances such as lysolecithin, pluronic polyols, polyanions, peptides, oil or hydrocarbon emulsions, keyhole limpet hemocyanins, and potentially useful human adjuvants such as N-acetyl-muramyl-L-threonyl-D-isoglutamine (thr-MDP), N-acetyl-nor-muramyl-L-alartyl-D-isoglutamine, N-acetylmuramyl-L-alanyl-D-isoglutaminyl-L-alanine-2-(1′-2′-dipalmitoyl-sn-glycero-3-hydroxyphosphoryloxy)-ethylamine, BCG (bacille Calmette-Guerin) and Corynebacterium parvum.

By way of further example, immunological adjuvants suitable for use in the immunogenic composition of the invention are Toll-like receptor (TLR) agonists such as TLR1, TLR2, TLR3, TLR4, TLR5, TLR6, TLR7, TLR8, TLR9 or TLR10 agonists, for example Hiltonol, SD101, Imiquimod, G100.

According to the invention, it is contemplated that the use of the immunogenic composition of the invention as a vaccine is prophylactic and/or therapeutic.

The immunogenic composition of the present invention may be used as vaccine for the treatment of an existing tumor disease or prophylactically to prevent the occurrence of this disease, particularly for the prevention and/or therapeutic treatment of a tumor, particularly a solid tumor, more particularly melanoma and/or melanoma residual disease.

A “prophylactic” treatment is a treatment administered to a subject who does not exhibit signs of a disease or exhibits only early signs for the purpose of decreasing the risk of developing the disease. The immunogenic composition disclosed herein as a vaccine can be given as a prophylactic treatment to reduce the likelihood of developing a tumor disease, particularly a solid tumor, more particularly melanoma, or to minimize the severity of the tumor disease, if developed.

A “therapeutic” treatment is a treatment administered to a subject who exhibits signs or symptoms of a disease for the purpose of diminishing or eliminating those signs or symptoms. In the treatment context, the administration of the immunogenic composition for use according to the invention can delay the progression and growth of a tumor disease, particularly a solid tumor, more particularly melanoma, by reducing the number of cancer cells and the primary tumor size, as well as inhibiting cancer cell infiltration into peripheral organs and tumor metastasis.

The immune status of the individual may be any of the following: the individual may be immunologically naive with respect to certain tumor-associated peptides present in the composition, in which case the compositions may be given to initiate or promote the maturation of an anti-tumor response. The individual may not currently be expressing anti-tumor immunity, but may have immunological memory, particularly T cell memory relating to a tumor-associated peptide comprised in the vaccine, in which case the compositions may be given to stimulate a memory response. The individual may also have active immunity (either humoral or cellular immunity, or both) to a tumor-associated peptide comprised in the vaccine, in which case the compositions may be given to maintain, boost, or maturate the response.

Within the therapeutic and/or preventative context, the immunogenic composition for use according to the invention may be applied to a tumor, for example melanoma, which is a tumor residual disease.

The term “tumour residual disease” is used herein to describe a stage of a tumour disease wherein clinical symptoms have disappeared or have largely disappeared (such as during treatment or after surgery) but a fraction of the cells originating from the treated diseased cells have survived the treatment or the surgery and/or developed tolerance to the drug(s) used in the treatment, and remain viable. These viable cells can be the origin of relapse.

The immunogenic composition of the invention is suitable to be administered as a vaccine for cancer therapy to any mammal, including human beings.

For the above mentioned therapeutic and preventative purposes, the immunogenic composition for use according to the invention may be administered alone or in combination with one or more other therapies against the tumor. Such other therapies include, for instance, a therapy with an immune checkpoint inhibitor, a chemotherapeutic agent, a biologicals, such as e.g. a nucleic acid therapy and antibodies neutralizing immune-modulator molecules, a target therapy such as e.g. an antibody therapy targeting the tumor, and an oncolytic virus therapy, and any combination of any of the foregoing combination therapies.

The immunogenic composition of the invention can be administered as a vaccine, for example by the intravenous, intradermal, intraperitoneal, subcutaneous or intramuscular routes.

Alternatively, the immunogenic composition for use according to the present invention, may be formulated and delivered in a manner to evoke an immune response at mucosal surfaces. Thus, the immunogenic composition may be administered to mucosal surfaces by, for example, the nasal, oral ocular, bronchiolar, or intrarectal routes.

The administration dose is selected as an amount which induces an immunoprotective response without significant adverse side effects, and is determined according to various factors, such as the morphology, hysto-type, size, and classification of the tumor to be treated, and can be determined by a person of ordinary skill in the art by using his/her normal knowledge.

In the light of the foregoing, a further aspect of the present invention relates to a method of preventing and/or treating melanoma in a subject in need thereof, said method comprising administering to the subject the immunogenic composition as above defined, wherein said administration induces an immune response against melanoma cells.

In one embodiment, the melanoma is a tumor residual disease.

Also included in the invention is a method of inducing an immune response against melanoma cells in a subject in need thereof, comprising administering to the subject an immunogenic composition as above defined.

In one embodiment, the induced immune response is a cell-mediated immune response, preferably involving the activation of cytotoxic T lymphocytes (CTLs).

As aforesaid, the one or more tumor-associated peptides according to the invention were isolated from the proteins secreted by bacteria-infected tumor cells. Particularly, the inventors observed that infection of melanoma cells with a bacterium such as e.g. Salmonella, induces the up-regulation and opening of membrane hemichannels and the release of proteasome-generated peptides in the extracellular milieu via the exacerbation of the unfolded protein response (UPR) pathway.

A preferred method for obtaining the one or more tumor-associated peptides comprises the steps of:
a) exposing a melanoma cell culture to at least one infectious agent and/or Pattern Recognition Receptor (PRR) agonist and/or inflammatory cytokine;
b) collecting the cell culture supernatant; and
c) isolating therefrom one or more tumor-associated peptides, wherein said one or more tumor-associated peptides comprise an amino acid sequence selected from the group consisting of SEQ ID Nos. 1-4 and 6-13 and fragments of said amino acid sequences SEQ ID NOs. 1-4 and 6-13 of at least 3 amino acids in length.

Within the context of the present description, the term “infectious agent”, means any biological material that can cause an infection and lead to a disease, including for example bacteria, viruses, fungi and parasites.

Preferably, the infectious agent is a bacterium, more preferably a Gram-negative bacterium, still more preferably a bacterium belonging to the Salmonella genus, even more preferably a non-virulent strain of Salmonella genus.

Infection by pathogenic microbes initiates a set of complex interactions between the pathogen and the host mediated by pattern recognition receptors. Pattern recognition receptors (PRRs) refer to germline-encoded receptors that are capable of recognizing molecules frequently found in pathogens (the so-called Pathogen-Associated Molecular Patterns—PAMPs), (PAMPs, Kawai T, Akira S. Toll-like receptors and their crosstalk with other innate receptors in infection and immunity. Immunity 2011; 34:637-50). PRRs activate downstream signaling pathways that lead to secretion of cytokines and activation of other host defense programs that are necessary for both innate and adaptive immune responses.

A PPR agonist refers to a compound (either natural or synthetic) that binds to PRR and triggers a response, thereby mimicking infectious agents.

Cytokines are key regulators of the immune response to infection. As it is known in the art, infection-triggered release of inflammatory cytokines leads to the opening of membrane hemichannels in different cell types (Saccheri F., et al; Bacteria Induced Gap Junctions in Tumor Favor Ag cross presentation and Antitumor Immunity. (2010). Sci Transl Med. 2010 Aug. 11; 2(44):44ra57).

Preferably, the inflammatory cytokine is gamma-IFN.

The melanoma cell used in the above-described method may be an established melanoma cell line or a melanoma cell isolated from a subject affected by melanoma.

Within the context of the present description, the expression “a cell line” is intended to mean a culture of a particular type of cell that can be reproduced indefinitely, whereas the expression “primary cell culture” is intended to mean a culture from a cell taken directly from a living organism, which is not immortalized.

The method for obtaining the one or more tumor associated peptides as above described is preferably carried out on a plurality of cell cultures of different melanoma cells. Where a plurality of melanoma cell cultures are used, the cultured cancer cells may derive from different melanoma patients or from cancer lesions in the same or different parts of a melanoma patient's body.

Based on this embodiment, through the above method are obtained tumor-associated peptides which are shared among melanoma patients and/or melanoma tumor types Preferably, in step c) of the method for obtaining the one or more tumor associated peptides, the collected cell culture supernatant is subjected to centrifugation and filtration to separate the liquid component.

The characterization of the peptides in the liquid component may be performed by any number of acceptable methods well known to those of skill in the art. In a preferred method, the characterization of the tumor-associated peptides is achieved by mass spectrometry analysis.

The following experimental section is provided purely by way of illustration and is not intended to limit the scope of the invention as defined in the appended claims. In the following experimental section reference is made to the appended drawings, wherein:

FIG. 1 shows that human melanoma cell lines, upon Salmonella infection, release immunogenic peptides able to prime healthy donor peripheral blood monocytes (PBMC). (A) Scheme of the experiment. Supernatant derived from 634-38 melanoma cells infected with Salmonella was collected and used to stimulate healthy donor HLA-A2⁺-PBMCs to generate cytotoxic T lymphocytes (CTLs, named as CTL-Vax). As positive control, PBMCs were stimulated with Mart-1₂₆₋₃₅ peptide (named as CTL-Mart1). CTLs were tested for their ability to recognize and kill HLA-A2 matched tumor cells. (B,D) Graphs showing the activation of cells by assessing IFN-gamma production and CD107a expression by flowcytometry. Either CTL-Vax (B) or CTL-Mart1 (D) were co-cultured for 5 hours in a ratio 1:1 with HLA-A2⁺ 624-38 melanoma cells, with 624-28 melanoma cells negative for HLA-A2, with HLA-A2⁺ melanoma SkMel24 and HLA-A2+ adenocarcinoma HT-29 cells, in the presence of Golgi inhibitor Brefeldin and CD107a antibody. To control the specificity of cells' activation an HLA-blocking antibody was added to the system. (C,E) Graphs showing the results of a Delfia assay conducted to assess cytotoxicity of either CTL-Vax (C) or CTL-Mart1 (E). Both 624-38 and 624-28 melanoma cells were loaded with Europium dye and co-cultured with effector either CTL-Vax or CTL-Mart1 cells at different target:effector ratio. The cytotoxic effect mediated by CTL-Vax is shown as percentage of specific Europium release. The Student's t-test was used for statistical analysis *p<0.05, **p<0.01, ***p<0.001, (n>4);

FIG. 2 shows that human melanoma cells release immunogenic peptides upon Salmonella infection. (A) Schematic illustration of the experimental strategy pursued to identify the candidate antigens responsible for the immunogenic effect of melanoma cell culture supernatants. (B) Table listing the candidate tumor-associated peptides identified according to the above procedure, along with their amino acid sequences, protein name and HLA-binding IC50. (C) The graphs show the immunogenic activity of the tumor-associated peptides by representing the IFN-gamma released by CTL-Vax upon stimulation with each single peptide. The tumor-associated peptides were synthesized, loaded on Thp1 at different concentrations (20 μM-1.25 μM) and used to stimulate CTL-Vax (ratio 1:1);

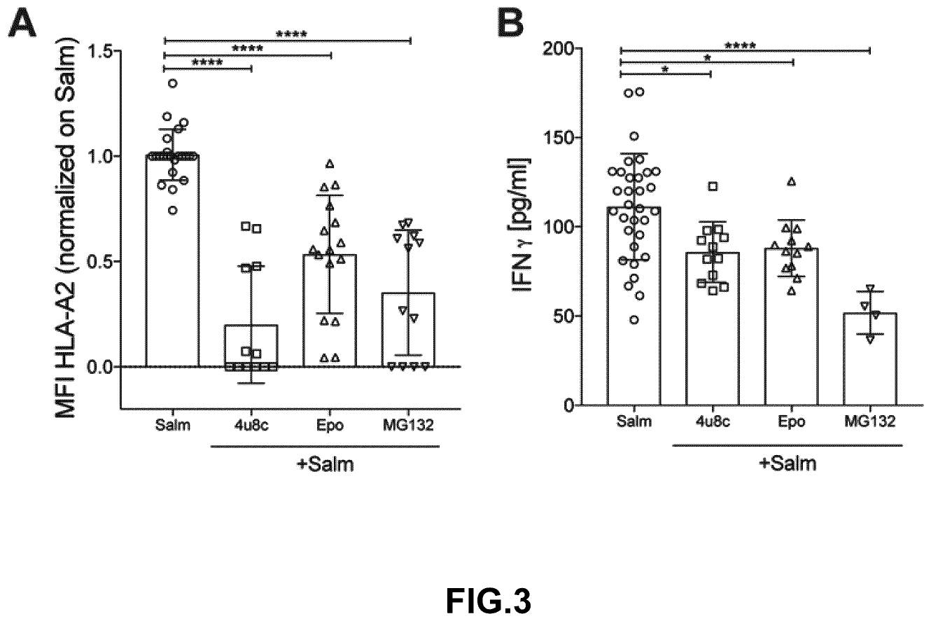

FIG. 3 shows that Salmonella infection induces a UPR-driven-release of proteasome-cleaved peptides by human tumor cells. (A) Bar graph showing the mean fluorescence intensity (MFI) of HLA-A*02:01 measured on the surfaces of T2 cells which were loaded for 2 hours with supernatants derived from Salmonella-infected 624-38 melanoma cells either in combination with UPR inhibitor 4μ8c or UPR inhibitor MG132 or Epoxomycin (Epo). (B) Bar graph showing the release of IFN-gamma by the human monocytic cell line Thp1 used as antigen presenting cell line and loaded with supernatants derived from Salmonella-infected 624-38 melanoma cells either in combination with UPR inhibitor 4μ8c or UPR inhibitor MG132 or Epoxomycin (Epo). Thp1 cells were co-cultured with CTLs Vax in a ratio 1:1. The release of IFN-gamma was measured after 72 hours by ELISA;

FIG. 4 shows that CTLs expanded with an immunogenic composition according to the invention comprising the tumor-associated peptides get activated only by tumor cells and not by primary melanocytes. The activation of CTLs was assessed by measuring IFN-gamma production by flowcytometry. (A) Scheme showing that CTL-Vax underwent two cycles of stimulation with the immunogenic composition of the invention in order to increase the frequency of peptides-specific CTLs, named as CTL-Vax-MIX. (B) The activation of CTLs was assessed by measuring IFN-gamma production by flowcytometry. Bar graphs show the results of CTL activation, by assessing the ability of both CTL-Vax (left) and CTL-Vax-MIX (right) to recognize target cells and release IFN-gamma. CTL-Vax and CTL-Vax-MIX were both co-cultured for 5 hours (ratio 1:1) with HLA-A2⁺ 624-38 melanoma cells, with 624-28 melanoma cells negative for HLA-A2, with HLA-A2⁺ melanocytes mel23 and HLA-A2 negative melanocytes mel41, in the presence of Golgi inhibitor Brefeldin and CD107a antibody. As a control of cell activation specificity, an HLA-blocking antibody was added to the system;

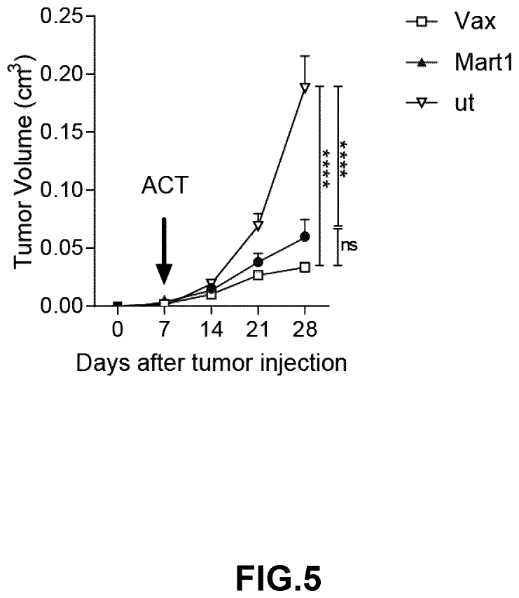

FIG. 5 shows that CTLs expanded with an immunogenic composition comprising the tumor-associated peptides according to the invention (CTL Vax) are able to kill tumor cells in vivo and exhibit a stronger effect than CTLs expanded with a known human melanoma antigen (Mart1). NSG mice were injected with 624-38 melanoma cells, left untreated (triangle), adoptively transferred with CTL-Vax (square) or with CTL-Mart1 (circle) at day 7 after tumor injection. Statistical analysis was evaluated using one-way ANOVA, *P<0.05, **P<0.01, ***P<0.001

### 1. MATERIALS AND METHODS

### Example 1.1: Cell Lines and Bacteria Strains

T2 cells (HLA-A0201 hybrid human cell line lacking TAP-2 (Hosken, Nancy A; Bevan, Michael J “Defective Presentation of Endogenous Antigen by a Cell Line Expressing Class I Molecules”. Science; Apr. 20, 1990; 248, 4953; Social Science Premium Collection pg. 367) were cultured in RPMI 1640 medium supplemented with 10% fetal bovine serum (FBS-South American), 2 mM glutamine, penicillin (100 U/ml), streptomycin (100 mg/ml), and 50 mM (3-mercaptoethanol (complete RPMI). Human melanoma cell lines 624-38 (HLA-A2 proficient, (Sabatino et al., “Conservation of Genetic Alterations in Recurrent Melanoma Supports the Melanoma Stem Cell Hypothesis”. (2008) Cancer Research (1). https://doi.org/10.1158/0008-5472.CAN-07-1939), 624-28 (HLA-A2 deficient), SkMel24 and the human colon cancer cell line HT29 were cultured in RPMI 1640 medium supplemented with 10% of FBS (North American), 2 mM glutamine, 100 U/ml penicillin, 100 mg/mL Streptomycin, 1% non-essential aminoacids (NeAA), 1% Sodium Pyruvate (NaPy). Peripheral blood monocytes (PBMCs) and cytotoxic T lymphocytes (CTLs) were cultured in RPMI supplemented with 5% human serum, 2 mM L-Glutamine, 100 U/ml penicillin, 100 mg/mL Streptomycin, 1% NeAA, 1% NaPy. Primary melanocytes (mel23, mel41) were grown in McCoy's 5A supplemented with 2 mM glutamin, 2% FBS, 100 U/ml penicillin, 100 mg/mL Streptomycin, Cholera Toxin 20 pM, Insulin 5 ug/ml (Roche), Hydrocortysone 0.5 ug/ml (SIGMA), Transferrin 5 ug/ml (Sigma), SCF 10 ng/ml (PEPROTECH), bFGF 1 ng/ml (PEPROTECH), Endothelin-1 10 nM (SIGMA).

In order to elucidate the Salmonella mechanism of action, the present inventors employed several inhibitors in their in vitro experiments, including: Heptanol (Sigma, 1 mM), hemichannel blocker; proteasome inhibitors: MG132 (Sigma, 20 μM) and Epoxomycin (Sigma, 500 nM); UPR-inhibitor 4μ8c (10 μM).

Vivofit® (Thyphoid vaccine live oral Ty21a) is a vaccine containing the attenuated strain of Salmonella enterica serovar Typhi Ty21a and is grown at 37° C. in Luria broth.

### Example 1.2: In Vitro Infection with Bacteria

Single bacterial colonies were grown overnight and restarted the next day to reach an absorbance at 600 nm of 0.6 corresponding to 0.6×10⁹ colony-forming units (CFUs)/ml. Murine and human melanoma cells were incubated with bacteria for 90 minutes, at a cell-to-bacteria ratio of 1:50, in the appropriate medium added with L-Glutamine without antibiotics. Cells were washed with medium and incubated in medium supplemented with gentamicin (50 mg/ml) for 18 hours to kill extracellular bacteria. At the end of the incubation cells were harvested and then lysed for protein analysis while supernatant was collected and filtered through 0.22 μm filter eliminate potentially still alive bacteria.

### Example 1.3: HLA-A*02:01-Peptide Binding Assay

To assess peptides enrichment in the supernatant from tumor cell cultures following Salmonella treatment, T2 cells were incubated overnight at 37° C. at 2×10⁵ cells/well in serum-free RPMI medium either with 100 μL of supernatant or with Mart-1₂₆₋₃₅ peptide (1 μM and 10 μM) as a positive control. After 2 or 18 hours, cells were blocked with mouse FcR block (BD), stained with BB7.2, an HLA-A2 conformation-specific mouse antibody (BD).

### Example 1.4: IFN-Gamma Assay for Peptide Recognition by CTLs

Human acute monocytic leukemia Thp1 cell line was loaded either with antigens at different concentration (20 uM-1.25 uM) or with conditioned medium and used to stimulate CTL-Vax (ratio 1:1). Cells supernatant was collected after 72 hours and IFN-gamma production was measured by ELISA (R&D). IFN-gamma released by CTLs co-cultured with unloaded Thp1 (spontaneous IFN-gamma release) was subtracted from IFN measured upon Ag-stimulation. Specificity of the activation was further assessed using an HLA-I pan blocker as control (10 ug/ml, Clone W6/32).

### Example 1.5: Antigen Specific-CD8⁺ T Cells Expansion from Healthy Donor PBMCs

Total PBMCs isolated from healthy HLA-A2⁺ donor were loaded either with supernatant derived by 2×10⁶ 624-38 cells treated with Salmonella or with 20 μM Mart-1₂₆₋₃₅ in tube on a rotator at 37° C. for 90 minutes, then plated in 24-well plates (2×10⁶ cells per well) in a final volume of 2 ml. From day 3 the recombinant IL-2 (proleukin, Novartis) was added at the final concentration of 20 U/mL. Cells were fed every 2-3 days with 20 U/mL IL-2 and restimulated every 10 day. At every restimulation, expanded lymphocytes were enriched in CD8⁺ T cells by magnetic column separation (Miltenyi). A total of 4×10⁵ of the isolated CD8⁺ T cells were plated with 2×10⁶ irradiated (10 Gy) HLA-A2⁺ PBMCs that were pulsed either with Mart-1 or with supernatant of 624-38 cells infected with Salmonella. To pulse PBMCs, these cells were incubated for 90 minutes at 37° C. in RPMI supplemented with the selected stimulus (Mart-1/cells' supernatant). After incubation, cells were washed twice and irradiated (10 Gy) before mixing with the CD8⁺ T cells.

### Example 1.6: CD107a Mobilization Assay

Effector cells included in the test were ex vivo expanded CD8⁺ T cells from human healthy donor PBMCs. Target cells were human melanoma cell lines (624-38, 624-28, SkMel24) and a human adenocarcinoma cell line (HT-29)). Effector cells and target cells were incubated in a tube in complete medium in ratio of 1:1 and 2:1. Anti-CD107a monoclonal antibody conjugated with APC (BD), brefeldin A 10 pg/ml (BD), GolgiStop containing monensin (BD), the HLA-I pan blocker as control (10 ug/ml, Clone W6/32) were added to the cells cultures in a final volume of 200 μl and incubated for 5 hours at 37° C. Cells were then stained with anti-CD3a, anti CD8 anti CD4 (BD) and then fixed in 4% PFA. Intracellular staining for IFN-gamma (BD) production was also performed in order to further assess T cell activation.

### Example 1.7: Delfia Cytotoxicity Test

Target cells (tumor cell lines) were collected, washed once and solved at concentration of 1×10⁶ cells/ml. A total of 2 ml of cells were loaded with 3 μl of Delfia-BATDA (DELFIA, Perkin Elmer) reagent, at 37° C. for 30 min. Following 4 washes with PBS and 1 wash with medium w/o serum, ^{5×103} cells were seeded in a v-bottom plate and incubated with effector PBMCs for 90 min in a humidified 5% CO₂ atmosphere at 37° C. The controls included in the experiment were: background (media without cells), spontaneous release (target cells without effector cells) and maximum release (lysed target cells). After incubation, cells were centrifuged for 5 minutes at 500 g and 20 μl of the supernatant was transferred to a flat-bottom plate and 180 μl of Europio solution were added. After 15 minutes of incubation at room temperature, the fluorescence was measured in the time-resolved fluorometer.

### Example 1.8: Mass Spectrometry Analysis

Sample preparation. Supernatants components were concentrated through the use of chromabond SPE C18 devices (Macherey-Nagel). Once eluted with 80% CH3CN in 0.1% Formic Acid (FA) solution, samples were dried by speed vacuum and solved in water. Proteins derived by 4×10⁶ cells were sonicated with Bioruptor 30″ ON+30″ OFF (2 cycles) and then loaded on AMICON Ultra 0.5 ml 10K (Millipore). Low molecular weight peptides were analyzed through n-LC-ESI-MS2 Q-Exactive HF.

Mass spectrometry. A total of 40 of each sample were loaded at max pressure of 900 bar on a LC-ESI-MS-MS quadrupole Orbitrap QExactive-HF mass spectrometer (Thermo Fisher Scientific). Peptides separation was achieved on a linear gradient from 95% solvent A (2% ACN, 0.1% formic acid) to 50% solvent B (80% acetonitrile, 0.1% formic acid) over 33 min and from 50 to 100% solvent B in 2 min at a constant flow rate of 0.25 μl/min on UHPLC Easy-nLC 1000 (Thermo Scientific) where the LC system was connected to a 23-cm fused-silica emitter of 75 μm inner diameter (New Objective, Inc. Woburn, MA, USA), packed in-house with ReproSil-Pur C18-AQ 1.9 μm beads (Dr Maisch Gmbh, Ammerbuch, Germany) using a high-pressure bomb loader (Proxeon, Odense, Denmark).

MS data were acquired using a data-dependent top 15 method for HCD fragmentation. Survey full scan MS spectra (300-1650 Th) were acquired in the Orbitrap with 60000 resolution, AGC target 3e6, IT20 ms. For HCD spectra, resolution was set to 15000 at m/z 200, AGC target 1e5, IT 80 ms; Normalized Collision energy 28% and isolation with 1.2 m/z. Technical replicates were conducted on the LC-MS-MS part of the analysis.

Database Searching. Data acquisition was controlled by Xcalibur 3.1. Mass spectra of the secretomes of human melanoma cell lines and patients were analyzed using the Mascot search engine. The precursor tolerance was set to 10 ppm and MS/MS tolerance was set to 20 ppm. The spectra were searched without enzyme specificity against the Uniprot human proteome database including isoforms (version 2016), methionine oxidation, deamidation (NQ), N-terminal acetylation and phosphorylation (STY) were set as variable modifications. Results were filtered for 1% FDR based on peptide spectrum matches (PSMs) using a target decoy approach with a randomized decoy database.

HLA binding prediction. NetMHCpan 4.0 Server was used to predict the HLA binding ability of the identified peptides, expressed as IC50 of HLA binding.

### Example 1.9: Adoptive T Cells Transfer

Groups of 7-week-old NSG mice were subcutaneously injected with 1×106 624-38 cells in their right flank. After one week, tumors became palpable and 8×106 antigen-specific CD8+ T cells, expanded in vitro with T Cell TransAct (Miltenyi Biotec) and resuspended in 200 μl PBS, were transferred by intravenous injection. Tumor growth was monitored by measuring the two visible dimensions with a caliper every 2 days.

### 2. RESULTS

### Example 2.1: Human Melanoma Cell Lines Release Immunogenic Peptides Upon Salmonella Infection that are Able to Prime Healthy Donor PBMCs

In order to verify whether the proteins secreted (secretome) by Salmonella-infected cells from human melanoma cell line 624-38 were immunogenic, the present inventors simulated what is happening in a general protocol of prophylactic vaccination for infectious disease, when immune system of healthy patients is stimulated with specific bacterial-epitopes. Briefly, CD8⁺ T cells were expanded from healthy donor peripheral blood mononuclear cells as depicted in FIG. 1A. These cells were enriched by means of a magnetic separation device and stimulated every two weeks with the secretome for a total of 4 rounds of stimulation.

In order to determine whether expanded CD8⁺ T cells (named CTL Vax) can recognize tumor cells, these cells were co-cultured with 624-38 melanoma cell, i.e. the cells from which the secretome was originated. The present inventors observed that CTL-Vax produced both IFN-gamma and CD107a molecules, indicating not only that CTL-Vax were able to recognize 634-38 tumor cells but also that these cells were degranulating in response to tumor cells (FIG. 1B). Moreover, the expression of the above-mentioned activation markers correlated with the killing ability of CTL-Vax (FIG. 1C). Importantly CTL-Vax recognized not only 624-38 cells but also SkMel24, another melanoma cell line (HLA-A2 proficient). In contrast, CTL-Vax did not recognize the adenocarcinoma HT-29 cells (HLA-A2 proficient). These results clearly show that CTL-Vax target tumor antigens that are shared among cells of the same tumor type, while at the same time CTL-Vax did not recognize melanoma 624-28 cells that are HLA deficient, thus validating that CTL-Vax activation was specifically induced by the recognition of an HLA-peptide complex. As a further control of the specificity of CTL-Vax activation, the inventors added an HLA-blocker antibody and observed consistently that CTL-Vax activation was totally impaired.

To further validate their analysis, the inventors used Mart-1₂₅₋₃₆, a known human melanoma antigen, to expand Mart-1-specific CTL from healthy donor PBMCs. Co-culture experiments were carried out as described above. As shown in FIG. 1C-D, CTL-Mart1 was activated and killed the 624-38 cell line that highly express Mart1 antigen. Of note, CTL-Mart1 did not kill SkMel24 cells that express the antigen at lower level.

Hence, the above illustrated results demonstrate that CTL-Vax guarantee the recognition of several shared tumor antigens giving an advantage in term of anti-tumor response and that healthy patients from which anti-tumor T cells were expanded had circulating lymphocytes that recognized them. This is an important aspect that grants the approach provided by the present invention and ultimately support its clinical translation.

### Example 2.2: Human Melanoma Cells Release Immunogenic Peptides Upon Salmonella Infection

In order to determine the nature of the peptides released by tumor cells following Salmonella infection, the inventors analyzed the secretome of the melanoma cell line 624-38 following the pipeline depicted in FIG. 2A. Briefly, the supernatant derived from 624-38 melanoma cells was filtered (cut-off 0.22 um), low molecular weight peptides (<10 kDa) were separated and analyzed by nLC-MS/MS. Selection criteria imposed for ms-signals were: MS/MS tolerance 20 mmu, peptide length >7aa, peptide tolerance 10 ppm. By applying a database search considering peptides with common post translational modifications (PTM), 135 peptides were found to be over-represented in the first condition.

Through the application of the same MS-based pipeline, the inventors evaluated the secretome of primary melanoma cells, which were infected or not with Salmonella. A total of 4212 peptides were identified derived from the melanoma of three human patients, of which 2054 were detected only in the Salmonella-infected secretomes. Among them, 28 peptides were present in all 3 patients' specimens.

For further selection, the peptides identified in the secretome of Salmonella-infected 624-38 cells were compared with those released by the three different patients-derived melanoma cells. Eleven peptides were selected, which are shared with at least one patient-derived cell line. Importantly, HLA-binding prediction (NetMHCpan 4.0 Server) scored all the selected peptides as HLA-binders with a predicted HLA-binding IC50 lower than 1000, specifically in the range of (150<IC50<840). Two additional peptides uniquely released by 624-38 cell line were selected for their HLA binding ability, for a total of thirteen candidates (FIG. 2B). The selected peptides were synthesized and loaded at different concentrations on Thp1 cells, a monocytic cell line chosen as antigen presenting cells. Of the thirteen candidate peptides, all except TMA7₄₇₋₄₄ (SEQ ID NO. 5) were able to induce IFN-gamma production by CTL-VAX confirming their presence within the Vax (supernatant derived by Salmonella-infected 624-38 tumor cells used to expand CTLs) as well as their immunogenic potential (FIG. 2C). Importantly, in the course of their experiments, the inventors also detected the known melanoma antigen Mart123-34 in the secretome of 624-38 cells. As shown in FIG. 2C, this melanoma antigen induced in the assay a lower IFN-gamma production by CTL-VAX than the novel identified peptides.

Based on the above results, the present inventors selected for further analysis the tumor-associated peptides having the amino acid sequences of SEQ ID Nos.:1-4 and 6-13 (as illustrated in FIG. 2B).

From a translational point of view, the identification of a plurality of tumor-associated peptides that are able, alone or in combination, to activate anti-tumor-CTLs has a great clinical impact, particularly considering that intra-tumoral heterogeneity can be efficiently targeted. Moreover, since the tumor-associated peptides of the invention were selected as being shared among different melanoma cells (FIG. 2A), and CTL-VAX target different melanoma cells (FIG. 1B), evidence were shown by the inventors that the selected tumor-associated peptides of the invention can be efficiently exploited for targeting shared-tumor antigens.

### Example 2.3: Salmonella Induces a UPR-Driven-Release of Proteasome-Cleaved Peptides by Human Tumor Cells

The present inventors investigated whether Salmonella infection of tumor cells may exacerbate the UPR response in these cells. Tumor cells need to afford plenty of stress stimuli at the level of endoplasmic reticulum (ER) due to a sustained proliferation, glucose shortage but also genomic instability; for these reasons tumor cells are characterized by a high basal level of unfolded protein response (UPR), that is now considered a sort of hallmark of such malignancies (Cerezo M. et al; (2016). “Compounds Triggering ER Stress Exert Anti-Melanoma Effects and Overcome BRAF Inhibitor Compounds Triggering ER Stress Exert BRAF Inhibitor Resistance”, Cancer Cell, 805-819 2016; Corazzari M. et al., (2017) “Endoplasmic Reticulum Stress, Unfolded Protein Response, and Cancer Cell Fate, 7(April), 1-11). With their studies, the inventors found that Salmonella infection of tumor cells exacerbated the UPR response in human melanoma cells, detectable both at gene and protein level as increased expression of XBP1s, CHOP and ATF4 (data not shown), without leading to an augmented cell death. Moreover, the inventors observed that the inhibition in 624-38 cells of IRE1a endoribonuclease and the subsequent splicing of XBP1, achieved through the use of 4μ8c compound, significantly impaired peptide release usually prompted by Salmonella infection (FIG. 3A) and that CTL-Vax had consistently a lower activation upon supernatant stimulation (FIG. 3B). In the same setting of experiments, the present inventors further observed that the inhibition of 20S-proteasome subunits with two different compounds, namely MG132 (MG) and Epoxomycin (Epo), affected both the release of the peptides of the invention (FIG. 3A) as well as CTL-Vax activation (FIG. 3B). In summary, although Salmonella infection does not alter proteasome function, the release of the peptides of the invention is partially dependent on proteasome activity.

### Example 2.4: CTL Expanded with a Plurality of the Peptides of the Invention Get Activated Only by Tumor Cells and not by Primary Melanocytes

The present inventors further assessed whether the selected immunogenic tumor-associated peptides are specifically presented by tumor cells, as they are normally undergoing a UPR response, and not by healthy melanocytic counterparts. In order to increase the frequency of the CD8⁺ T cells specific for the peptides of the invention, CTL-VAX underwent two cycles of stimulation with irradiated HLA-A2 PBMCs pulsed with a composition comprising the twelve immunogenic tumor-associated peptides (SEQ ID NOs. 1-4 and 6-13), and were named as CTL-Vax-MIX (FIG. 4A). Afterwards, both CTL-Vax and CTL-Vax-MIX were co-cultured with 624-38 (HLA-A2 proficient), 624-28 (HLA-deficient), primary melanocytes mel23 (HLA-A2 proficient) and primary melanocytes mel41 (HLA-A2 deficient). The inventors observed that CTL-Vax expressed IFN-gamma in the presence of the HLA-A2 proficient mel23 while, importantly, the CTL-Vax-MIX did not (FIG. 4B).

These results clearly indicate that the tumor-associated peptides of the invention that expanded CTL-Vax-MIX are presented effectively only by tumor cells and not by normal melanocytes, thereby further granting the safety of their clinical application.

### Example 2.5: Cytotoxic Potential In Vivo of Ex-Vivo Expanded CTL-Vax CD8⁺ T Cells

In order to test the fitness of the ex-vivo expanded CTL-Vax CD8⁺ T cells, the present inventors assessed their cytotoxic potential in vivo. NSG mice were subcutaneously inoculated with 624-38 melanoma cells and adoptively transferred with (i) CTLs expanded in vitro with an immunogenic composition according to the invention comprising the twelve tumor-associated peptides (CTL Vax) or (ii) CTLs expanded in vitro with the known human melanoma antigen Mart-1 (CTL-Mart1). Notably, tumor growth was inhibited by CTL-Vax that showed a significantly stronger tumor control effect than CTL-Mart1 (FIG. 5).

As illustrated in the above Experimental examples, the inventors have demonstrated that the immunogenic composition of the invention can prime cytotoxic T cells in vitro that are effective through a HLA-dependent fashion in killing melanoma cell lines in vitro (FIGS. 1 and 4). These further results show that CTL-Vax kill tumor cells in vivo after adoptive T cell transfer (FIG. 5). Expanded T cells are fit enough to kill melanoma cells when xenotrasplanted in vivo and this is an important and positive indication of the T cell response that the immunogenic tumor-associated peptides could generate in vivo upon vaccination.

1. An isolated antigen-presenting cell (APC), which carries on the cell surface one or more tumor-associated peptides comprising an amino acid sequence selected from the group consisting of SEQ ID NOs. 1-4 and 6-13, fragments of said amino acid sequences SEQ ID NOs. 1-4 and 6-13 of at least 3 amino acids in length, and any combination thereof.

2. The isolated antigen-presenting cell (APC) according to claim 1, which carries on the cell surface a tumor-associated peptide comprising SEQ ID NO. 1, a tumor-associated peptide comprising SEQ ID NO. 2, a tumor-associated peptide comprising SEQ ID NO. 3 and a tumor-associated peptide comprising SEQ ID NO. 4.

3. The isolated antigen-presenting cell (APC) according to claim 1, which carries on the cell surface a tumor-associated peptide comprising SEQ ID NO. 1, a tumor-associated peptide comprising SEQ ID NO. 2, a tumor-associated peptide comprising SEQ ID NO. 3, a tumor-associated peptide comprising SEQ ID NO. 4, a tumor-associated peptide comprising SEQ ID NO. 7, a tumor-associated peptide comprising SEQ ID NO. 8, a tumor-associated peptide comprising SEQ ID NO. 11, a tumor-associated peptide comprising SEQ ID NO. 12 and a tumor-associated peptide comprising SEQ ID NO. 13.

4. The isolated antigen-presenting cell (APC) according to claim 1, which carries on the cell surface a tumor-associated peptide comprising SEQ ID NO. 1, a tumor-associated peptide comprising SEQ ID NO. 2, a tumor-associated peptide comprising SEQ ID NO. 3, a tumor-associated peptide comprising SEQ ID NO. 4, a tumor-associated peptide comprising SEQ ID NO. 6, a tumor-associated peptide comprising SEQ ID NO. 7, a tumor-associated peptide comprising SEQ ID NO. 8, a tumor-associated peptide comprising SEQ ID NO. 9, a tumor-associated peptide comprising SEQ ID NO. 10, a tumor-associated peptide comprising SEQ ID NO. 11, a tumor-associated peptide comprising SEQ ID NO. 12 and a tumor-associated peptide comprising SEQ ID NO. 13.

5. The isolated antigen-presenting cell (APC) according to claim 1, which is a dendritic cell.

6. An immunogenic composition comprising:
(i) one or more tumor-associated peptides comprising an amino acid sequence selected from the group consisting of SEQ ID NOs. 1-4 and 6-13, fragments of said amino acid sequences SEQ ID NOs. 1-4 and 6-13 of at least 3 amino acids in length, and any combination thereof;
(ii) one or more isolated nucleic acid sequences encoding the tumor-associated peptide(s) of (i);
(iii) one or more expression vectors comprising the nucleic acid sequence(s) of (ii); and/or
(iv) one or more antigen-presenting cells (APC) according to claim 5, and a pharmaceutically acceptable vehicle.

7. The immunogenic composition according to claim 6, which comprises a tumor-associated peptide comprising SEQ ID NO. 1, a tumor-associated peptide comprising SEQ ID NO. 2, a tumor-associated peptide comprising SEQ ID NO. 3 and a tumor-associated peptide comprising SEQ ID NO. 4.

8. The immunogenic composition according to claim 6, which comprises a tumor-associated peptide comprising SEQ ID NO. 1, a tumor-associated peptide comprising SEQ ID NO. 2, a tumor-associated peptide comprising SEQ ID NO. 3, a tumor-associated peptide comprising SEQ ID NO. 4, a tumor-associated peptide comprising SEQ ID NO. 7, a tumor-associated peptide comprising SEQ ID NO. 8, a tumor-associated peptide comprising SEQ ID NO. 11, a tumor-associated peptide comprising SEQ ID NO. 12 and a tumor-associated peptide comprising SEQ ID NO. 13.

9. The immunogenic composition according to claim 6, which comprises a tumor-associated peptide comprising SEQ ID NO. 1, a tumor-associated peptide comprising SEQ ID NO. 2, a tumor-associated peptide comprising SEQ ID NO. 3, a tumor-associated peptide comprising SEQ ID NO. 4, a tumor-associated peptide comprising SEQ ID NO. 6, a tumor-associated peptide comprising SEQ ID NO. 7, a tumor-associated peptide comprising SEQ ID NO. 8, a tumor-associated peptide comprising SEQ ID NO. 9, a tumor-associated peptide comprising SEQ ID NO. 10, a tumor-associated peptide comprising SEQ ID NO. 11, a tumor-associated peptide comprising SEQ ID NO. 12 and a tumor-associated peptide comprising SEQ ID NO. 13.

10. The immunogenic composition according to claim 6, optionally further containing an adjuvant, for use as a vaccine.

11. The immunogenic composition for use as a vaccine according to claim 10, in a combination therapy with an immune checkpoint inhibitor, a chemotherapeutic agent, a biologicals, target therapy and/or an oncolytic virus.

12. (canceled)

13. (canceled)

14. A method of preventing and/or treating melanoma in a subject in need thereof, said method comprising administering to the subject an immunogenic composition as defined in claim 6, wherein said administration induces an immune response against melanoma cells.

15. The method according to claim 14, wherein the melanoma is a tumor residual disease.

16. A method of inducing an immune response against melanoma cells in a subject in need thereof, said method comprising administering to the subject an immunogenic composition as defined in claim 6.

17. The method according to claim 16, wherein the immune response is a cell-mediated immune response.

18. The method according to claim 17, wherein the cell-mediated immune response involves the activation of cytotoxic T lymphocytes (CTLs).
